(19) United States
(12) Patent Application Publication
(10) Pub. No.: US 2021/0381037 A1
(43) Pub. Date: Dec. 9, 2021

(54) APPARATUS AND METHOD FOR HIGH THROUGHPUT PARALLEL NUCLEIC ACID SEQUENCING ON SURFACES OF MICROBEADS

(71) Applicant: ARDENT BIOMED GUANGDONG CO., LTD, Guangzhou (CN)

(72) Inventor: David Xingfei DENG, Mountain View, CA (US)

(21) Appl. No.: 17/311,113

(22) PCT Filed: Jun. 25, 2019
(86) PCT No.: PCT/CN2019/092820

§ 371 Date: Jun. 4, 2021

(51) Int. Cl. C12Q 1/6837; C12Q 1/6874; B01L 3/00

(52) U.S. Cl. CPC C12Q 1/6837; C12Q 1/6874; B01L 2300/0636; B01L 3/502761; B01L 3/502715

(57) ABSTRACT

Method and apparatus for nucleic acid sequencing are provided. The method includes providing a microbead disposed in one reaction well and immobilized with two capturing oligonucleotides with different sequences, immobilizing nucleic acid templates on the microbead via annealing between the templates and the capturing oligonucleotides, amplifying the immobilized nucleic acid templates and producing a population of template clones annealed with sequencing primers. The method further includes sequentially disposing different types of nucleotide trisphosphates, detecting, by ion-sensitive field-effect transistors, ion concentration change in the reaction wells in response to incorporation of one of the nucleotide trisphosphates at 3′ end of sequencing primers, when the nucleotide trisphosphates is complementary to a corresponding nucleotide in the template clones, and sequencing the template clones by repeating the sequentially disposing and the detecting. A method for producing single-stranded nucleic acid template clones on a reaction well array is also provided.

### FIELD OF THE DISCLOSURE

The present disclosure relates generally to the field of nucleic acid sequencing, and more particularly, relates to apparatus and method for high throughput parallel nucleic acid sequencing on surfaces of microbeads.

### BACKGROUND

Nucleic acid sequencing includes determination of the order of nucleotides, the chemical building blocks that make up nucleic acid. In a typical arrangement, DNA or RNA samples are fragmented, enriched, sequenced and analyzed to obtain the sequence information, which could be utilized in a broad range of biological and pharmaceutical applications including diagnostics of genetic diseases, drug trials and pharmacogenomics, as well as other applications such as evolutionary biology and forensics. Especially since the completion of the Human Genome Project in 2001, a rapid expansion of knowledge about human DNA and genetic variation has been initiated, which further boosts the development of nucleic acid sequencing technologies.

A number of sequencing techniques have been developed and some of them are commercialized. One of them is based upon a fluorescent imaging platform. Each type of deoxynucleoside triphosphate (e.g., dATP, dCTP, dTTP and dGTP) is labeled with fluorescently labeled reversible terminators. During the sequencing process, each sequencing cycle only allows a single dNTP added to the growing oligonucleotide strand. Concurrently with the single dNTP incorporation, the fluorescently labeled reversible terminator is imaged to identify a corresponding base in the template strand, and the terminator is subsequently cleaved to allow the incorporation of next dNTP. This method requires a delicate fluorescent imaging platform as well as at least four dNTPs labeled with different fluorescently labeled reversible terminators as building blocks, which cause high instrument cost and more importantly, significant reagent cost leading to high expense per run, especially for sequencing with a large genome size.

Alternatively, ion semiconductor sequencing is another sequencing technique based on detection of hydrogen ions released from incorporation of dNTPs into a growing nucleotide strand. Hydrogen ions are natural byproducts of polymerase-catalyzed nucleotide extension reactions. When a single dNTP is incorporated into the growing nucleotide strand, it releases one hydrogen ion which can be detected by an ion-sensitive field-effect transistor to generate an electronic signal. If homopolymer repeats are present, multiple hydrogen ions are released, corresponding to proportional increase in the electronic signals. Prior to sequencing process in the ion semiconductor sequencing method, DNA templates are attached onto micrometer-sized beads which are compartmentalized into water-oil emulsion droplets containing PCR reaction mixture. In the aqueous water-oil emulsion, each of the droplets containing the micrometer-sized bead functions as a PCR microreactor that amplifies the attached DNA template fragments. However, emulsion PCR is a time-consuming process requiring multiple steps (forming and breaking emulsion, PCR amplification, enrichment, etc.). Further, the emulsion breaking and bead washing are usually carried out by centrifugation, during which the beads may aggregate causing sample loss. It is also relatively inefficient since only around two thirds of the emulsion microreactors actually contain one bead while other emulsion droplets are empty. Therefore, an extra step may be required to separate empty emulsion droplets leading to more potential inaccuracies.

The disclosed apparatus and method for nucleic acid sequencing are directed to solve one or more problems set forth above and other problems.

### BRIEF SUMMARY OF THE DISCLOSURE

One aspect of the present disclosure provides a method for nucleic acid sequencing. A plurality of microbeads is provided each disposed in one of a plurality of reaction wells and modified with at least two capturing oligonucleotides with different sequences. A plurality of single-stranded nucleic acid templates is immobilized on surfaces of the plurality of microbeads via annealing between the plurality of single-stranded nucleic acid templates and the at least two capturing oligonucleotides. Each single-stranded nucleic acid template includes two regions complementary to the different sequences of the at least two capturing oligonucleotides, respectively. The immobilized plurality of single-stranded nucleic acid templates is amplified and a population of single-stranded nucleic acid template clones is produced on the surfaces of the plurality of microbeads. The population of single-stranded nucleic acid template clones is annealed with a plurality of sequencing primers. Different types of nucleotide trisphosphates are sequentially disposed into the plurality of reaction wells. The different types of nucleotide trisphosphates are known. An ion concentration change in the plurality of reaction wells in response to incorporation of one of the different types of nucleotide trisphosphates at 3′ end of one of the sequencing primers is detected by one or more ion-sensitive field-effect transistors (ISFETs), when the one of the different types of nucleotide trisphosphates is complementary to a corresponding nucleotide in the population of single-stranded nucleic acid template clones. The population of single-stranded nucleic acid template clones is sequenced by repeatedly performing the sequentially disposing of the different types of nucleotide trisphosphates and the detecting, by the one or more ISFETs, of the ion concentration change in the plurality of reaction wells.

Another aspect of the present disclosure provides a method for producing single-stranded nucleic acid template clones on a reaction well array. The reaction well array including a plurality of reaction wells is provided. A plurality of microbeads is disposed in the plurality of reaction wells, and at least two capturing oligonucleotides with different sequences are immobilized on a surface of each of the plurality of microbeads. A solution including a plurality of single-stranded nucleic acid templates is added into the plurality of reaction wells. Each of the single-stranded nucleic acid templates includes two regions complementary to the different sequences of the at least two capturing oligonucleotides, respectively. The plurality of single-stranded nucleic acid templates is immobilized on surfaces of the plurality of microbeads via annealing between the nucleic acid templates and the at least two capturing oligonucleotides. A number of the single-stranded nucleic acid templates immobilized on a surface of each microbead via the annealing is less than or equal to a pre-determined value, and the pre-determined value is one. The immobilized plurality of single-stranded nucleic acid templates is amplified, thereby generating a plurality of double-stranded nucleic acid template clones. The plurality of double-stranded nucleic acid template clones is denatured and a population of single-stranded nucleic acid template clones is produced on the surfaces of the plurality of microbeads.

Another aspect of the present disclosure provides an apparatus for nucleic acid sequencing. The apparatus includes a sensor array, including a plurality of ion-sensitive field-effect transistors (ISFETs) configured to provide at least one output signal corresponding to a concentration or presence of one or more ions proximate thereto; a flow cell including an input, an output and a flow chamber, the flow chamber being in fluidic connection with an opening of each reaction well of an array of reaction wells, a fluidics delivering unit, configured to be in fluidic connection with the input of the flow cell, and configured to deliver at least one of the to-be-sequenced nucleic acid template and different types of known nucleotide trisphosphates, in a direction from the input to the output, to the reaction chamber. A plurality of microbead is disposed in the array of reaction wells. At least two capturing oligonucleotides with different sequences are immobilized on a surface of each of the microbeads, and the different sequences of the at least two capturing oligonucleotides are complementary to two regions of a to-be-sequenced nucleic acid template. Each of the reaction wells is associated with one of the plurality of ISFETs in the sensor array, and the one of the plurality of ISFETs is configured to provide the at least one output signal in response to ion concentration change in each of the reaction wells. The ion concentration change corresponds to incorporation of one of the different types of nucleotide trisphosphates at 3′ end of a sequencing primer annealed to the to-be-sequenced nucleic acid template, when the one of the different types of nucleotide trisphosphates is complementary to a corresponding nucleotide in the to-be-sequenced nucleic acid template.

Other aspects of the present disclosure can be understood by those skilled in the art in light of the description, the claims, and the drawings of the present disclosure.

### BRIEF DESCRIPTION OF THE DRAWINGS

To describe the technical solutions in the embodiments of the present disclosure more clearly, the following briefly introduces the accompanying drawings used for describing the embodiments. Apparently, the accompanying drawings in the following description show merely some embodiments of the present disclosure, and a person skilled in the art may still derive other drawings from these accompanying drawings without creative efforts.

FIG. 1 illustrates a block diagram of an exemplary nucleic acid sequencing apparatus according to various embodiments of the present disclosure;

FIG. 2 illustrates a diagram of a portion of the fluidics delivering unit and the detecting unit in an exemplary nucleic acid sequencing apparatus according to various embodiments of the present disclosure;

FIG. 3 illustrates a diagrammatic workflow of single-stranded nucleic acid template amplification and sequencing performed by an exemplary nucleic acid sequencing apparatus according to various embodiments of the present disclosure;

FIG. 4 illustrates a diagrammatic workflow of single-stranded nucleic acid template amplification performed by an exemplary nucleic acid sequencing apparatus according to various embodiments of the present disclosure;

FIGS. 5A-5B illustrate two flow charts of digital capturing and amplification of single-stranded nucleic acid templates to produce nucleic acid template clones, performed by an exemplary nucleic acid sequencing apparatus according to various embodiments of the present disclosure;

FIG. 6 illustrates a diagrammatic workflow of nucleic acid template sequencing performed by another exemplary nucleic acid sequencing apparatus according to various embodiments of the present disclosure; and

FIG. 7 illustrates a diagrammatic workflow of nucleic acid template amplification and sequencing performed by another exemplary nucleic acid sequencing apparatus according to various embodiments of the present disclosure.

### DETAILED DESCRIPTION

The following describes the technical solutions in the embodiments of the present disclosure with reference to the accompanying drawings. Apparently, the described embodiments are merely some but not all the embodiments of the present disclosure. Other embodiments obtained by a person skilled in the art based on the embodiments of the present disclosure without creative efforts shall fall within the protection scope of the present disclosure.

The present disclosure provides an apparatus for nucleic acid sequencing, configured to determine the sequence information of the nucleic acid (e.g., DNA or RNA) in a sample. The nucleic acid sequencing apparatus may be used to determine the sequences of individual genes, larger genetic regions (e.g., clusters of genes), full chromosomes, or whole genome of an organism. Further, the nucleic acid sequencing apparatus may also be used in RNA sequencing and methylation sequencing by identifying methylation patterns in the genome. The nucleic acid sequencing apparatus according to the present disclosure may function in a variety of manners depending upon different applications, including sequencing-by-synthesizing as well as other sequencing methods such as sequencing by ligation or pyrosequencing, which will not be limited in the present disclosure. In an exemplary arrangement which will be described in greater detail below according to the present disclosure, based on genome size of the nucleic acid in a sample and the amount of the sample available to be sequenced, the nucleic acid may be firstly fragmented, followed by 5′ and 3′ adaptor ligation and denaturing processes to create a sample library containing single-stranded nucleic acid templates. Further, the sample library may be disposed into a reaction chamber of the nucleic acid sequencing apparatus, in which these single-stranded nucleic acid templates may further be amplified to create a population of single-stranded nucleic acid template clones, the sequences of which may be determined by the subsequent sequencing process.

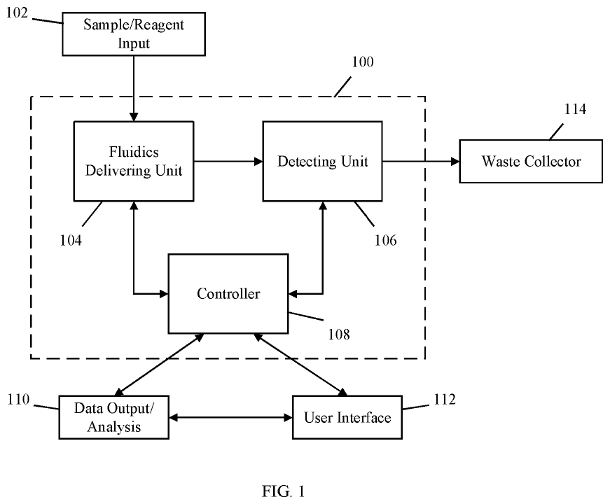

FIG. 1 illustrates a block diagram of an exemplary nucleic acid sequencing apparatus according to various embodiments of the present disclosure. The exemplary nucleic acid sequencing apparatus 100 may include a fluidics delivering unit 104, a detecting unit 106 and a controller 108. The fluidics delivering unit 104 may be configured to deliver nucleic acid sample solution to be sequenced, reagent solutions containing one or more of dNTPs, polymerase and electrolytes, as well as rinse or wash solutions to the detecting unit 106. The fluidics delivering unit 104 may include one or more of solution containers, valves, pumps and to tubing configured to store and transfer solutions to the detecting unit 106 in a configurable manner. The detecting unit 106 may include one or more flow cells in fluidic connection with a plurality of micrometer-sized reaction wells. Each of the flow cells may include an input, an output and a reaction chamber. The input of the flow cell may be connected to the fluidics delivering unit 104, such that the solutions from the fluidics delivering unit 106 may be transferred into the reaction chamber through the input. The output of the flow cell may be connected to a waste collector 114. The reaction chamber may form a flow path in fluidic connection with openings of the plurality of reaction wells, such that the molecules flowing through the flow path, in a direction from the input to the output of the flow cell, may freely diffuse into the reaction wells, and further, the molecules in the reaction wells (e.g., excessive reagents) may diffuse out of the reaction well and exit from the output of the flow cell. In one embodiment, one or more of micrometer-sized beads may be disposed in each of the reaction wells. In one embodiment, each of the reaction wells may have a sensor suitable for detecting characteristics of chemical or enzymatic reactions occurring within the reaction well, and convert the characteristics change to analog or digital signals as output. Optionally, a sample/reagent input 102 may be connected with the fluidics delivering unit, provide sample and reagent solutions to the fluidics delivering unit 102. In another embodiment, the detecting unit may further be connected to a waste collector 114 which contains reagent solutions and wash solutions exited from the flow cell of the detecting unit 106. More details regarding the fluidics delivering unit and the detecting unit will be described in greater detail below.

The controller 108 of the exemplary nucleic acid apparatus may be connected with the fluidics delivering unit 102 and the detecting unit 103. The controller 108 may include a general purpose or application-specific computer system configured to control the delivery of fluidics, acquire signals outputted from the detecting unit, process and output the output signals, as well as other functions as desired. For example, the controller may configure the order of the reagents being delivered to the detecting unit and preset the parameters of the fluidics delivery, including flow rate, flow duration, etc. The controller may also be configured to acquire and process signal outputted from the detecting unit 106 into formats recognizable by a data output/analysis unit 110, and a user interface 112.

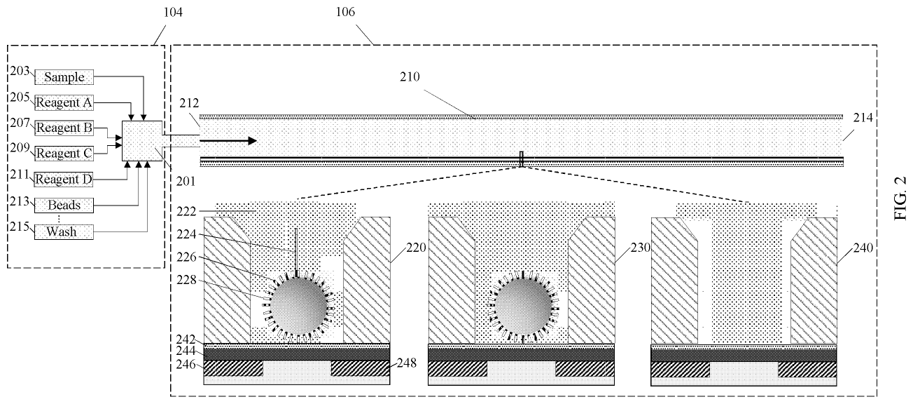

FIG. 2 illustrates a diagram of a portion of the fluidics delivering unit and the detecting unit in an exemplary nucleic acid sequencing apparatus according to various embodiments of the present disclosure. With reference to FIG. 2, The detecting unit 106 may include one or more flow cells. Each of the flow cells includes an input 212 in fluidic connection with the fluidics delivering unit 104, an output 214 in connection with the waste collector 114, and a flow chamber 210 in fluidic connection with opening portions of a plurality of reaction wells. For example, FIG. 2 illustrates an enlarged cross-sectional view of three reaction wells 220, 230 and 240. The opening portion 222 of the reaction well 220 may be in fluidic connection with the flow chamber 210. In one embodiment, the flow cell may be a microfluidic device. The plurality of reaction wells may be disposed on a microfluidic chip made of semiconductors, polymers or metals by microfabrication or nanofabrication techniques known to one of ordinary skill in the art, for which the present disclosure will not described in detail herein. The microfluidic chip may further include a set of micro flow channels in connection with the reaction wells. Through the fluidic connection between the opening portions of the reaction wells and the micro flow channels, the molecules flowing through the reaction chamber may diffuse in and out of the reaction wells. For example, the plurality of reaction wells may be arranged in a matrix to form a reaction well array. In one embodiment, the microfluidic chip may include 10 thousand to 200 million reaction wells, and the size, e.g., a diameter, of each reaction well may be below 20 micrometers. In another embodiment, the microfluidic chip may include 1 million to 6 million reaction wells, and the size, e.g., diameter, of each reaction well may be below 5 micrometers. For example, a proton chip may be used, including 160 million reaction wells. In another example, a PGM 318 chip may be used, including 16 million reaction wells. It should be noted that the number, size, shape, volume or depth of the reaction wells in the reaction well array as well as the method used for manufacturing the reaction well array may be determined in accordance with various applications for which the present disclosure will not intend to be limiting.

In one embodiment, one or more beads may be disposed in each of the reaction wells. The micrometer-sized beads (also called as microbeads) may have a size of less than 20 micrometers. For example, the size of the microbeads may be ranged from 0.5-20 μm, 1-10 μm, or 0.5-5 μm, which may be compatible with the size of the reaction wells. In one embodiment, each of the reaction wells may contain a single microbead, while the existence of the microbead may not affect the fluidic connection between the flow chamber and the reaction wells. The material of the microbeads may be selected from polystyrene, silica, hydrogel, glycidal methacrylate and magnetic materials. When the magnetic microbeads were used in the present disclosure, the magnetic material (e.g., iron) may be encapsulated to ensure that it may not interfere with DNA polymerase and other enzymes. The surface of the microbeads may be modified with one or more of reactive groups configured for adjusting the surface properties of the microbeads and for facilitating the immobilization of biomolecules. For example, the reactive groups may include one or more of carboxylic acid group, amine group, amide group, hydroxyl group, hydrazide group, thiol group, epoxy group, primary aliphatic amine group, aromatic amine group, aldehyde group, and vinyl benzyl chloride group.

Nucleic acids may be covalently attached to the surface of the microbeads. In one embodiment, oligonucleotides (e.g. oligos) may be immobilized on the surface of the microbeads. These oligonucleotides may function as probes for capturing single-stranded nucleic acid templates in the sample solution flowing over the microbeads disposed in the reaction wells, where each of the single-stranded nucleic acid templates may contain complementary sequences (e.g., adaptor sequences at 3′ and 5′ ends of the single-stranded nucleic acid templates) to the oligonucleotide probes. In one embodiment, at least two capturing oligonucleotides with different sequences may be immobilized on the surface of the microbeads, and the sequences of the capturing oligonucleotides may be complementary to different regions of the nucleic acid templates. With reference to FIG. 2, for example, two types of capturing oligos 226 and 228 may be immobilized on the surface of the microbeads disposed in the reaction wells 220 and 230. The sequence of the capturing oligo 228 may be complementary to adaptor sequence at one end of the single-stranded nucleic acid template 224, while the sequence of the capturing oligo 226 being complementary to the adaptor sequence at the other end of the template. Through the annealing between the capturing oligos and the nucleic acid template flowing over and diffusing into the reaction wells, the single-stranded nucleic acid template may be captured by the capturing oligos and therefore, immobilized on the surface of the microbeads.

In one embodiment, only one single-stranded nucleic acid template may be captured by the capturing oligos and immobilized on the surface of the microbeads. Subsequently, the single-stranded nucleic acid template may be amplified to generate a dense amount of nucleic acid template clones on the surface of the microbeads. In one embodiment, the immobilized capturing oligos may function as primers during the amplification process, therefore, the density of the capturing oligos may affect the density of the generated template clones. With reference to FIG. 2, for example, the microbead disposed in the reaction well 220 may be captured with a nucleic acid template strand 224. When the density of the immobilized capturing oligos on the surface of the microbeads was substantially low, a majority of the microbeads (e.g., the microbead disposed in the reaction well 230 of FIG. 2) may not be captured with any nucleic acid template after the sample solution containing the template was flushed over the reaction wells, which may reduce the efficiency of the template capturing, amplification and the following sequencing. On the other side, when the density of the immobilized capturing oligos was substantially higher, the generated template clones may be too dense at the bottom portions of the reaction wells, which may negatively affect the accuracy and efficiency of the following sequencing step.

With further reference to FIG. 2, each of the reaction wells may be connected to at least one sensor such that one or more of the characteristics of chemical or enzymatical reactions occurring within the reaction wells may be detected and measured by the at least one sensor. For example, the detecting unit 106 may include a sensor array including a plurality of sensors. Each of the sensors in the sensor array may correspond to a single reaction well in the reaction well array. In one embodiment, the sensors in the sensor array may be chemical field-effect transistor (ChemFET) measuring concentration change of one or more chemicals (e.g. a reactor or a product) in a reaction solution. In another embodiment, the sensor may be an ion-sensitive field-effect transistor (ISFET) detecting ion concentration change in a reaction solution. As shown in FIG. 2, for example, the ISFET sensors may be located below the reaction wells 220/230/240. In particular, the ISFET sensor may include a source electrode 246, a drain electrode 248, an ion-sensitive layer 242 and optionally a gate electrode 244. The ion-sensitive layer 242 may be in fluidic connection with the reaction solution disposed in the reaction wells and detect the change in ion concentration on the surface of or in proximity to the ion-sensitive layer, which may cause a change in the current between the source electrode 246 and the drain electrode 248. The current change corresponding to the ion concentration change within the reaction well may be directly outputted or further converted to voltage or impedance change as output signal, depending upon specific applications for which the present disclosure will not describe herein. It should be noted that the configurations of the ISFET with top-gate structure in FIG. 2 are for illustrative purposes only, any other suitable structures such as bottom-gate structures may be encompassed within the present disclosure. In other embodiments, the ISFET may have other configurations based upon specific applications for which the present disclosure will not limit herein.

In one embodiment, the ion concentration change may be caused by the newly generated ions as byproducts of the chemical or enzymatic reactions occurring on the surface of the microbeads. The newly generated ions may diffuse within the reaction wells, e.g., on the surface of or in proximity to the ion-sensitive layer of the ISFET sensors and detected by the ISFETs. As such, the ISFET sensors may be configured to detect the ion concentration change on the surface of the microbeads.

In accordance with the aforementioned embodiments, the ISFET sensor in the exemplary sequencing apparatus may further include a reference electrode in fluidic connection with the reaction wells, providing a same voltage to all of the reaction wells. In one embodiment, the reference electrode may be one or more micro electrodes integrated on the sensor array. The sensor array may be fabricated on a circuit-connected substrate where the circuit is connected to the controller 104. As such, the signal change detected by each sensor may be collected and processed by the controller 104. In one embodiment, the sensor array and the reaction well array may be integrated on a same semiconductor chip.

In one embodiment, the sensor in the exemplary nucleic acid apparatus may be a pH-sensitive ISFET detecting concentration change of the hydrogen ions. For example, the pH-sensitive ISFET may detect the generation of hydrogen ions from a polymerase-catalyzed oligonucleotide extension reaction within the reaction wells. In one embodiment, the polymerase-catalyzed oligonucleotide extension reaction may occur on the surface of the microbeads, where the beads may be disposed in the reaction wells. In particular, with each dNTP incorporating into a growing nucleotide strand immobilized on the surface of the microbeads, a hydrogen ion, as a natural byproduct of the dNTP incorporation, may be generated and diffused in proximity to the ion-sensitive layer of the ISFET and detected by the ISFET. Accordingly, the concentration of the released hydrogen ions may be proportional to the concentration of the incorporated dNTP. In another embodiment, four types of dNTPs may be sequentially delivered to the reaction wells in a pre-determined order, one type of dNTP at a time. In the presence of a DNA polymerase and a primer annealed to the nucleic acid template to form a primer-template duplex on the surface of the microbeads, only a dNTP complementary to a next base in the template strand may be incorporated at 3′ end of the primer strand and release a hydrogen ion which may be detected by the ISFET and generate a positive signal output. The other three types of dNTPs which are not complementary to the base may be flushed out of the reaction well without generating a positive signal. Based on the positive signal generated with the incorporation of the complementary dNTP, the base on the template strand may be identified. When this reaction cycle is repeated, the sequence of the entire nucleic acid template may be identified in such sequencing-by-synthesizing manner.

With regard to the fluidics delivering unit 104, it may include one or more of solution containers, valves, pumps and tubing, for storing and transferring solutions to the detecting unit 106 in a configurable manner. As shown in FIG. 2, for example, the fluidic delivering unit 104 may include a valve unit 201 with pumps and tubing, configured to deliver sample solution stored in a sample solution container 203, as well as reagent A (e.g., a solution containing dATP, DNA polymerase and electrolyte) stored in reagent container 205, reagent B (e.g., a solution containing dGTP, DNA polymerase and electrolyte) stored in reagent container 207, reagent C (e.g., a solution containing dTTP, DNA polymerase and electrolyte) stored in reagent container 209, reagent D (e.g., a solution containing dCTP, DNA polymerase and electrolyte) stored in reagent container 211, microbead solution stored in microbead solution container 213, and wash solution (e.g., weakly buffered solution) stored in wash solution container 215. In some of the embodiments, a portion or all of the reagent solutions and wash solutions may be integrated into one or more cartridges which could be installed into the exemplary nucleic acid apparatus.

Under the control of the controller 108, and the delivered solutions may vary depending upon the working stages of the nucleic acid sequencing apparatus. For example, during a sequencing-by-synthesis stage as described in the aforementioned embodiment, the delivered solution may sequentially deliver reagents (e.g., reagents A, B, C and D), followed by a washing solution. That is, the four types of dNTPs, one at a time, may be sequentially delivered to the reaction chamber of the detecting unit in the pre-determined order, e.g., dATP, dGTP, dTTP, dCTP, dATP, dGTP, dTTP, dCTP and so forth. After each delivery of single type of dNTP, the reaction chamber may be exposed with wash solutions to remove excessive dNTP. Optionally, a dNTP-destroying solution (e.g., apyrase), after the washing, may be delivered to eliminate any residual dNTP remaining in the reaction chamber and reaction wells. It should be noted that the aforementioned order of the dNTP addition is for exemplary purposes only, for which the present disclosure will not intend to be limiting.

In one embodiment, during a microbead loading stage, a solution containing oligonucleotide-modified microbeads may be delivered to the reaction chamber, such that the microbeads may be diffused and disposed in the reaction wells. For example, each of the reaction wells may contain a single microbead. In order to realize this, the concentration of the microbeads may be adjusted base upon a total number of the reaction wells. In one embodiment, a total number A of the microbeads in the flow chamber may be adjusted according to a total number B of the reaction wells, where A≤2×B. In another embodiment, the total number A of the microbeads in the flow chamber may be less than or equal to the total number B of the reaction wells, that is A≤B. In some of the optional embodiments, the total number A of the microbeads in the flow chamber may be less than or equal to 90%, 80% or 70% of the total number B of the reaction wells, that is A≤0.9×B, A≤0.8×B or A≤0.7×B.

In some of the optional embodiments, the loading of the oligonucleotide-modified microbeads may be repeated. As illustrated in FIG. 2, after the initial loading of the microbeads, a portion of the reaction wells may be disposed with a single microbead (e.g., reaction wells 220 and 230) and other portions of the reaction wells may remain empty (e.g., reaction well 240). In order to maximize a ratio between a number of reaction wells containing microbeads and a total number of reaction wells, the loading of the microbeads may be repeated. That is, the solution stored in the microbead solution container 213 may be repeatably delivered to the reaction chamber, settled in the reaction chamber for a preset duration and exited from the output of the flow cell. As such, a majority of the reaction wells may be disposed with microbeads which may improve the efficiency and effectiveness of the subsequent amplification and sequencing on the surface of the microbeads.

In another embodiment, during a sample loading stage, the single-stranded nucleic acid templates may be delivered to the reaction chamber such that the template may be captured onto the surface of the microbeads for subsequent amplification. A number of the plurality of single-stranded nucleic acid templates immobilized on the surface of the microbeads via the annealing may be less than or equal to a pre-determined value. In one embodiment, the pre-determined value may be one, that is, a single nucleic acid template may be immobilized on the surface of each microbead. To realize the single nucleic acid template immobilization, in one embodiment, the fluidics delivering unit 104 may deliver a sample solution containing a low concentration of nucleic acid template in a per-determined flow rate for pre-determined duration, such that a low concentration of single nucleic acid template may diffuse into each of the reaction wells and immobilized on the surface of the microbeads. Additionally, during other working stages, the fluidics delivering unit 104 may deliver cleavage solution for cleaving the linkers on the capturing oligos, and denaturing solution (e.g., containing NaOH) for removing one strand of the double-stranded amplified nucleic acid templates to form a population of single-stranded nucleic template clones on the surface of the reaction wells.

The present disclosure also provides a method for nucleic acid sequencing. The method may include the following steps: providing a plurality of microbeads, where each of the plurality of microbeads may be disposed in one of a plurality of reaction wells and immobilized with at least two capturing oligonucleotides with different sequences, and immobilizing a plurality of single-stranded nucleic acid templates on surfaces of the plurality of microbeads via annealing between the plurality of single-stranded nucleic acid templates and the at least two capturing oligonucleotides, where each of the single-stranded nucleic acid templates may include two regions complementary to the different sequences of the at least two capturing oligonucleotides, respectively, as well as amplifying the immobilized plurality of single-stranded nucleic acid templates and producing a population of single-stranded nucleic acid template clones on the surfaces of the plurality of microbeads, where the population of single-stranded nucleic acid template clones may be annealed with a plurality of sequencing primers. The method for nucleic acid sequencing may further include sequentially disposing different types of nucleotide trisphosphates into the plurality of reaction wells where the different types of nucleotide trisphosphates are known, and detecting, by one or more ion-sensitive field-effect transistors (ISFETs), an ion concentration change in the plurality of reaction wells in response to incorporation of one of the different types of nucleotide trisphosphates at 3′ end of one of the sequencing primers, when the one of the different types of nucleotide trisphosphates is complementary to a corresponding nucleotide in the population of single-stranded nucleic acid template clones; and sequencing the population of single-stranded nucleic acid template clones by repeating the sequentially disposing of the different types of nucleotide trisphosphates and the detecting, by the one or more ISFETs, of the ion concentration change in the plurality of reaction wells.

A workflow of the exemplary method for nucleic acid sequencing will be described in greater detail below. It should also be noted that the exemplary sequencing method according to the present disclosure may be carried out by the exemplary apparatus for nucleic acid sequencing. Alternatively, the exemplary sequencing method according to the present disclosure may be carried out by other apparatus, for which the present disclosure will not intend to be limiting.

In one embodiment, the single-stranded nucleic acid template may firstly be pre-treated before disposing into the reaction wells. In particular, nucleic acid (e.g. genomic DNA) may be extracted and fragmented to generate a collection of double-stranded nucleic acid fragments of which the sequences may be of interest to obtain sequence information. After or concurrently with the fragmentation process, both 3′ and 5′ ends of the double-stranded nucleic acid fragments may be ligated with two adaptors, respectively, followed by a denaturing process to form a plurality of single-stranded nucleic acid templates, where each template may include two adaptors ligated at 3′ end and 5′ end, respectively. It should be noted that other sample preparation methods may also be used for which the present disclosure will not intend to limit. In one embodiment, the nucleic acid extraction and sample preparation may approximately take 90-120 minutes.

After the sample preparation process, the sample solution containing a plurality of single-stranded nucleic acid templates may be disposed into the fluidics delivering unit 104 through the sample/reagent input 102. The nucleic acid templates may flow in the reaction chamber and diffused into the opening portion of the plurality of reaction wells. In accordance with the aforementioned embodiments, capturing oligos with sequences complementary to the 3′ and 5′ end adaptors on the single-stranded template, respectively, may be immobilized in the reaction wells.

In some of the optional embodiments, the reaction wells may be disposed with a plurality of microbeads, that is, each of the plurality of microbeads may be disposed in one reaction well and modified with at least two capturing oligonucleotides (capturing oligoes) with different sequences. Through the annealing between the adaptor at 3′ or 5′ end of the template and the capturing oligo, the single-stranded nucleic acid template may be immobilized on the surface of microbeads. Accordingly, before or concurrently with disposing the sample solution into the flow chamber, the method for nucleic acid sequencing may further include disposing a solution containing the microbeads into the flow chamber, such that the microbeads may diffuse into the reaction wells. In some of the optional embodiments, the disposing of the solution containing the microbeads may be repeated in a configurable manner until a majority or all of the reaction wells may each contain a microbead, thereby improving the efficiency of the capturing and immobilization of the template on the surface of the microbeads, as well as the subsequent amplification and sequencing of the template.

To realize the delivery of the microbeads in the reaction wells, the parameters of the fluidics delivering unit 104, e.g., the flow rate and duration of the solution containing the microbeads may also be adjusted to pre-determined settings. After the microbead solution flows from the input of the flow path and filled the reaction chamber, it may be settled in the reaction chamber for a pre-determined duration, such that the microbeads may diffuse in proximity to and located within the reaction wells.

In one embodiment, a number of the single-stranded nucleic acid templates immobilized on the surface of each microbead via the annealing may be less than or equal to a pre-determined value. For example, the pre-determined value may be one. In other words, for each of the microbeads, it may be immobilized with a single nucleic acid template, alternatively it may contain none of the templates, resulting in digital capture of the nucleic acid templates, that is, either 1 or 0 single-stranded nucleic acid template may be immobilized on the surface of each microbead. As shown in FIG. 2, with the sample solution containing the nucleic acid templates flowing in the reaction chamber, the reaction well 220 containing a capturing oligo-modified microbead may be immobilized with a single nucleic acid template 224, while the reaction well 230 may contain another capturing oligo-modified microbead with no immobilized template, and the reaction well 240 may contain neither microbead nor template. The reaction well including a single template may be associated with a positive signal output (e.g. a binary readout of 1) while the reactions wells including none of the templates may only show background signal configured as a negative signal output (e.g., a binary readout of 0). The digital capturing of the single-stranded nucleic acid template may possess a variety of advantages. For example, after the amplification of a single nucleic acid template, a dense cluster of template clones may be formed on the surface of the microbeads, bring in high reproducibility and precision to the following sequencing step. Additionally, the amplification of a single nucleic acid template through the digital capturing may realize absolute quantification without the need for running standard curves or reference, significantly improving the accuracy and sensitivity of the amplification and the sequencing step.

To realize the digital capturing of the nucleic acid template, in one embodiment, the concentration of the sample solution containing the templates may be properly adjusted to a pre-determined value. For example, the concentration of the template in the sample solution may be diluted so that a number of single-stranded nucleic acid templates per volume in the reaction chamber may be less than a total number of the reaction wells. In one embodiment, the number of the single-stranded nucleic acid templates per volume in the reaction chamber may be less than or equal to 90% of the total number of the reaction wells. Optionally, the number of the single-stranded nucleic acid templates per volume in the reaction chamber may be less than or equal to 80% of the total number of the reaction wells. Optionally, the number of the single-stranded nucleic acid templates per volume in the reaction chamber may be less than or equal to 70% of the total number of the reaction wells. In one embodiment, when the number of the nucleic acid templates per volume in the reaction chamber may be less than the total number of the reaction wells, the polyclonal capturing of the template into the reaction wells may be significantly reduced based upon Poisson distribution. That is, the ratio of the reaction wells immobilized with more than one single-stranded nucleic acid template to a total number of the reaction wells immobilized with nucleic acid templates may be reduced. For example, the polyclonal capturing of the nucleic acid template may be reduced to below 20%, when the number of the nucleic acid templates per volume in the reaction chamber may be≤90%,≤80% or≤70% of the total number of the reaction wells. The low ratio of the polyclonal capturing of the template in the reaction wells may be removed during data analysis, such that it may not cause interference in the subsequent sequencing process.

Furthermore, to realize the digital capturing of the nucleic acid templates, the parameters of the fluidics delivering unit 104, e.g., the flow rate and duration of the sample solution containing the templates may also be adjusted to pre-determined settings. After the sample solution flows from the input of the flow path and filled the reaction chamber, it may be settled in the reaction chamber for a pre-determined duration, such that the template may diffuse in proximity to the capturing oligos immobilized in the reaction wells.

In another embodiment, the sample solution containing the single-stranded nucleic acid templates may be partitioned to generate a plurality of small droplets, each of the droplets including 1 or 0 of the templates. For example, a 20 microliter of sample solution may be partitioned into 20,000 nanoliter-sized droplets. Subsequently, these droplets may be injected into the fluidics delivering unit 104 through the sample/reagent input 102, flowing in the reaction chamber 210. With the concentration of the droplets in the reaction chamber as well as one or more of the flow parameters adjusted, a single droplet may be disposed into each of the reaction wells. The template within the single droplet may be released and hybridized with the capturing oligos in the reaction well to realize the digital capturing of the template.

The method for nucleic acid sequencing may further include the steps of amplifying the immobilized plurality of single-stranded nucleic acid templates and producing a population of single-stranded nucleic acid template clones on the surfaces of the plurality of microbeads, where the population of single-stranded nucleic acid template clones may be annealed with a plurality of sequencing primers. Further, the method may include sequentially disposing different types of nucleotide trisphosphates into the plurality of reaction wells wherein the different types of nucleotide trisphosphates are known, and detecting, by one or more ISFETs, an ion concentration change in the plurality of reaction wells in response to incorporation of one of the different types of nucleotide trisphosphates at 3′ end of one of the sequencing primers, when the one of the different types of nucleotide trisphosphates is complementary to a corresponding nucleotide in the population of single-stranded nucleic acid template clones, and sequencing the population of single-stranded nucleic acid template clones by repeating the sequentially disposing of the different types of nucleotide trisphosphates and the detecting, by the one or more ISFETs, of the ion concentration change in the plurality of reaction wells. One or more embodiments in accordance with the aforementioned steps will be described as follows.

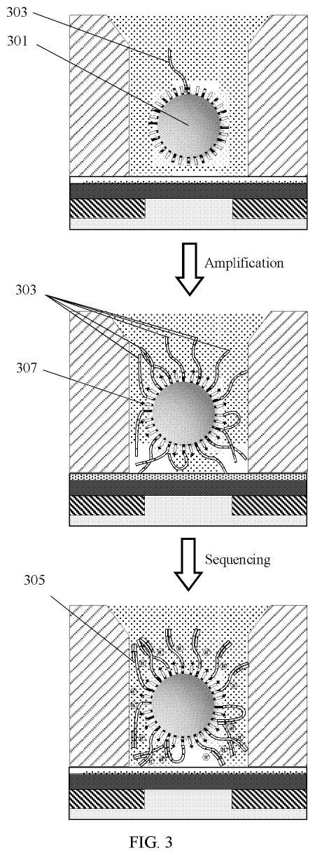

FIG. 3 illustrates a diagrammatic workflow of single-stranded nucleic acid template amplification and sequencing performed by an exemplary nucleic acid sequencing apparatus according to various embodiments of the present disclosure. After the digital capture of the template, a single nucleic acid template 303 may be immobilized on the surface of the microbead 301. Subsequently, in the presence of amplification reagents containing DNA polymerase, dNTPs and electrolytes, the single nucleic acid template may further be amplified to generate a plurality of nucleic acid template clones on the surface of the microbead. For example, the nucleic acid template may be amplified by performing isothermal amplification in the presence of one or more DNA polymerases suitable for the specific amplification method and dNTPs as building blocks.

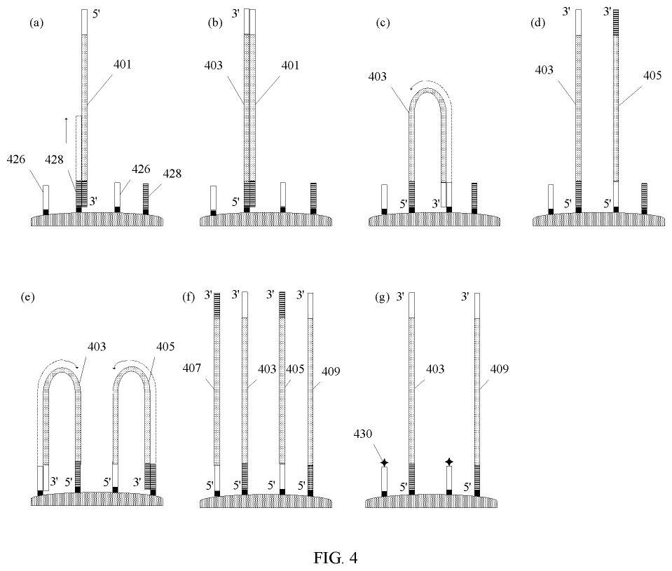

In one embodiment, bridge amplification may be performed to amplify the single nucleic acid template immobilized on the surface of the microbead. FIG. 4 illustrates a diagrammatic workflow of single-stranded nucleic acid template amplification performed by an exemplary nucleic acid sequencing apparatus according to various embodiments of the present disclosure. In one embodiment with reference to step (a) of FIG. 4, a single nucleic acid template 401 including two adaptor sequences at 3′ and 5′ ends, respectively, may be captured by annealing to one of the capturing oligos 428 immobilized on the surface of the microbead, where the adaptor sequences at 3′ end may be complementary to the sequence of the capturing oligo 428. The capturing oligo 428 may be extended in the presence of the nucleic acid template 401, DNA polymerase and dNTPs to form an elongated oligonucleotide strand 403 (see (b) of FIG. 4). After denaturing and removing of the original nucleic acid template 401, the extended elongated oligonucleotide strand 403 which may have a complementary sequence as the removed nucleic acid template 401, may bend over and approach the surface of the reaction well. The adaptor sequence at 3′ end of the oligonucleotide strand 403 may be complementary to and therefore, annealed to another capturing oligo 426. The capturing oligo 426 may extend using the oligonucleotide strand 403 as a template, to form an elongated oligonucleotide strand 405 having the same sequence as the nucleic acid template 401 (see (c) of FIG. 4). After denaturing (see (d) of FIG. 4), the two oligonucleotide strands 403 and 405 may further bend over and anneal to the capturing oligos 426 and 428, respectively, to form new oligonucleotide strands 407 and 409 using the capturing oligos as primers (see (e) and (f) of FIG. 4). The aforementioned steps may be rapidly repeated, thereby forming a dense amount of double-stranded nucleic acid template clones may be formed on the bottom of the reaction wells. For each amplification cycle, one capturing oligo modified on the surface of the microbead may be occupied. With the completion of the bridge amplification, a majority of the capturing oligos may be occupied. For example, after the amplification process, a microbead having a diameter of 5 micrometers and modified with 1,000-1,000,000 capturing oligoes may include 1,000-2,000,000 template clones on the surface of the microbead.

In another embodiment of the present disclosure in accordance with (g) of FIG. 4, part of the amplified nucleic acid templates may be retained on the surface of the microbeads, while the other part of the amplified nucleic acid templates may be cleaved and the corresponding capturing oligos and free 3′ ends of the template strands may be blocked. For example, the amplified nucleic acid template 403 and 409, having 5′ ends in proximity to the surface of the microbead and 3′ ends away from the surface of the microbead, may be retained. The amplified nucleic acid template 405 and 407, which are the reverse strands of the template 403 and 409, may be cleaved, with corresponding capturing oligos being blocked by blocking reagents 430. As such, all of the amplified single stranded nucleic acid templates may have the same direction. Since the above amplification process starts with a single nucleic acid template, the precision and reproducibility of the amplification may be significantly improved, thereby increasing the accuracy of the following sequencing step. It should be noted that the bridge amplification method in the aforementioned embodiment is for exemplary purposes only. Other amplification methods in accordance with suitable DNA polymerase and other reagents used in specific protocols, for which the present disclosure will not intend to be limiting.

As described above, the digital capture of the template in the reaction well may also result in reaction wells containing none of the template. For example, some of the reaction wells may contain microbeads but with no immobilized template, and some of the reaction wells may remain empty without any microbead or template. Accordingly, the digital capturing of the nucleic acid template within the reaction well may need to be monitored, thereby determining a loading rate of the reaction wells. In particular, the load rate may be a ratio between a number of the reaction wells each containing one microbead immobilized with one single-stranded nucleic acid template and a total number of reaction wells. In one embodiment, when an ISFET sensor was associated with each of the reaction wells, the amplification of the nucleic acid template may also be monitored by output signals of the ISFET sensors in response to ion concentration change during the template amplification. The loading rate may further be determined by monitoring the output signals of the ISFET sensors during the amplification of the nucleic acid template. For example, the sample solution containing the single-stranded nucleic acid templates may be firstly flushed into the reaction chamber for digital capturing of the template in each of the reaction wells. When a single template is immobilized on the surface of the microbead, the ISFET sensor associated with the reaction well may be configured to monitor the amplification process of the single template by measuring the concentration change of the released hydrogen ions during the amplification, and outputting signals (e.g. a binary signal of one). For empty reaction wells without nucleic acid template, the associated ISFET sensors may only show background signal as a negative output signal (e.g. a binary output of zero). As such, the loading rate of the reaction wells may be determined by quantifying the number of reaction wells with a binary output of one.

In accordance with the aforementioned embodiments, the present disclosure also provides a method for producing single-stranded nucleic acid template clones on a reaction well array, including the steps of providing the reaction well array including a plurality of reaction wells, where a plurality of microbeads may be disposed in the plurality of reaction wells, and at least two capturing oligonucleotides with different sequences may be immobilized on a surface of each of the plurality of microbeads and adding a solution including a plurality of single-stranded nucleic acid templates into the plurality of reaction wells. In one embodiment, each of the single-stranded nucleic acid templates includes two regions complementary to the different sequences of the at least two capturing oligonucleotides, respectively. Accordingly, the plurality of single-stranded nucleic acid templates may be immobilized on surfaces of the plurality of microbeads via annealing between the nucleic acid templates and the at least two capturing oligonucleotides, and a number of the single-stranded nucleic acid templates immobilized on a surface of each microbead via the annealing may be less than or equal to a pre-determined value. For example, the pre-determined value may be one. The method for producing single-stranded nucleic acid template clones on the reaction well array may further include amplifying the immobilized plurality of single-stranded nucleic acid templates, thereby generating a plurality of double-stranded nucleic acid template clones; and denaturing the plurality of double-stranded nucleic acid template clones and producing a population of single-stranded nucleic acid template clones on the surfaces of the plurality of microbeads. It should also be noted that the exemplary method for producing single-stranded nucleic acid template clones on the reaction well array according to the present disclosure may be carried out by the exemplary apparatus for nucleic acid sequencing. Alternatively, the exemplary method may be carried out by other instruments or performed on other platforms, for which the present disclosure will not intend to be limiting.

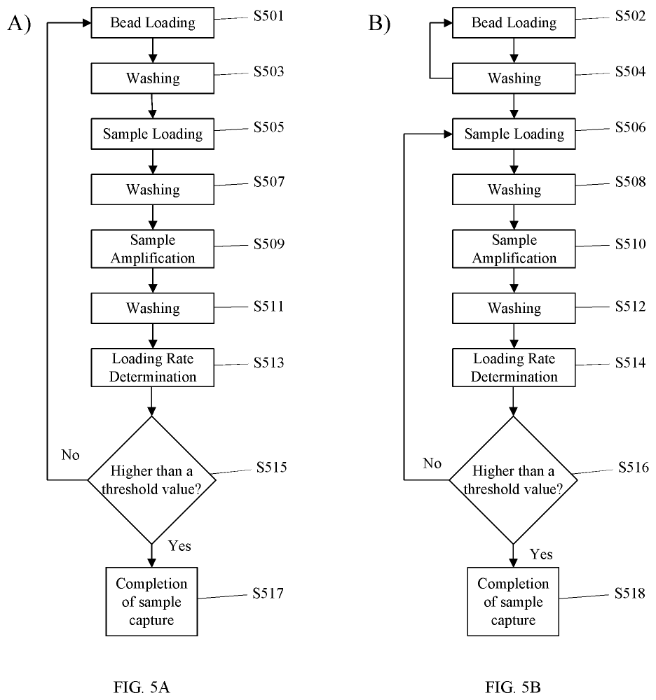

FIGS. 5A-5B illustrates two flow charts of digital capturing and amplification of single-stranded nucleic acid templates to produce nucleic acid template clones, performed by an exemplary nucleic acid sequencing apparatus according to various embodiments of the present disclosure. As illustrated in FIG. 5A, a loading cycle for digital capturing of the single-stranded nucleic acid template may include S501 of microbead loading, S505 of sample loading as well as S509 of sample amplification, each followed by washing (S503, 507 and S511) respectively.

During S505, the sample amplification, that is, the amplification of the single nucleic acid template immobilized on the surface of the microbeads may be monitored, thereby determining a loading rate for the currently loading cycle in S513. When the loading rate was lower than a pre-determined threshold value, the loading cycle may be repeated, including repeating S501 of microbead loading, S505 of sample loading as well as S509 of sample amplification, each followed by washing step. When the determined loading rate was higher than or equal to the threshold value, the loading may be completed and ready for sequencing. In particular, during S501 of bead loading, the solution containing a pre-determined concentration of microbeads may be disposed into the reaction chamber through the fluidic delivering unit and flushed out by the washing solution during S503. After or concurrently with the bead loading, during S505, a sample solution containing a pre-determined concentration of single-stranded nucleic acid template may be disposed into the reaction chamber and flushed out by the wash solution during S507. A portion of the reaction wells may be disposed with microbeads, where a single nucleic acid template may be immobilized on the microbead, while another portion of the reaction wells may only have microbead without immobilized template, or remain empty without bead or template. In accordance with the aforementioned embodiments, the immobilized nucleic acid template may be amplified during S509 and monitored for the amplification process, followed by the S511 of washing. Concurrently with or after S509, a loading rate corresponding to the current loading cycle may be determined (S509) and compared with the pre-determined threshold value (S515). In one embodiment, the production of nucleic acid template clones on a reaction well array may approximately take 40-70 minutes.

The loading cycle may be repeated until the loading rate exceeds the pre- determined threshold value to indicate completion of sample capture (see e.g., S517 of FIG. 5A and S518 of FIG. 5B). As illustrated in FIG. 5A, the loading cycle may be performed by repeating the steps of S501, S505 and S509, that is, the bead loading may be included in the repeatable loading cycle. For example, a cycle of sample capture may be repeated twice, in particular, bead loading→sample loading→sample amplification→bead loading→sample loading→sample amplification, until the calculated loading rate is higher than or equal to the pre-determined threshold value.

Alternatively, FIG. 5B illustrates another flow chart of an exemplary loading cycle. As illustrated in one embodiment according to FIG. 5B, S502 of bead loading may be performed and optionally repeated until a majority or all of the reaction wells are disposed with a microbead prior to sample loading. Further, S506 of sample loading and S510 of sample amplification may be proceeded while the loading rate may be determined after or concurrently with the sample amplification (S514). In one embodiment according to FIG. 5B, a cycle of sample capture may be repeated twice, in particular, bead loading→bead loading→sample loading→sample amplification→sample loading→sample amplification, until the calculated loading rate is higher than or equal to the pre-determined threshold value (S516 and S518).

Optionally, each cycle of sample capture may be repeated three times or more. Additional washing steps may be needed between two cycles. The pre-determined threshold value and cycle numbers may be pre-set by an operator through the user interface 112. Furthermore, the concentration of the template in the sample solution used for each loading cycle may be the same. Optionally, the fluidics delivering unit may deliver a small portion of the sample solution in a configurable manner while the remaining sample solution stored in the sample solution container 203 and ready to be delivered for next loading cycle. Alternatively, the concentration of the nucleic acid template in the sample solution used for the initial loading cycle may vary from the subsequent loading cycles. In another optional embodiment, the fluidics delivering unit may deliver a small portion of microbead solution in a configurable manner while the remaining microbead solution stored in the microbead solution container 213 and ready to be delivered for next cycle.

In accordance with the aforementioned embodiments, the loading rate may be determined in different ways. For example, the ISFET associated with each reaction well may be configured to provide at least one output signal in response to ion concentration change within the reaction wells, where the ion concentration change may correspond to the amplification of the immobilized plurality of nucleic acid templates on the surface of the microbeads. As such, the digital capturing followed by the amplification of the nucleic acid template on the surface of the microbeads may be monitored. On one side, the time-consuming emulsion PCR which includes the steps of formation of water-in-oil emulsion and breaking of the emulsion, may be avoided. In some embodiments of the present disclosure, all of the reactions may be performed in water phase environment to avoid any water-in-oil emulsion. On the other side, the amplification process of the single nucleic acid template on the surface of the microbeads may be monitored without the addition of dye or other probe molecules. That is, additional detection platforms, e.g., a fluorescent imaging system may no longer be needed in the method or the apparatus for nucleic acid sequencing according to the present disclosure. Instead, the ISFET sensors associated with reaction wells may be configured to monitor the hydrogen ions generated from the amplification of the nucleic acid template, thereby simplifying the method and the apparatus for nucleic acid sequencing significantly.

Referring back to FIG. 3, with the completion of amplification of the single-stranded template in the reaction wells, a dense amount of double-stranded nucleic acid templates may be formed on the surface of the microbeads. After denaturing the duplex and removing reverse strands, a plurality of single-stranded templates 303 may remain immobilized on the surface of the microbeads and ready for sequencing, while other oligos blocked by the blocking molecules 307. During the sequencing step, in the presence of sequencing primers 305 annealed to the nucleic acid template clones, DNA polymerase and known dNTPs, by detecting one or more characteristics of the sequencing primer extension reaction, the incorporated dNTPs at 3′ end of the growing sequencing primer may be identified in the sequencing-by-synthesis manner, as described below in greater details.

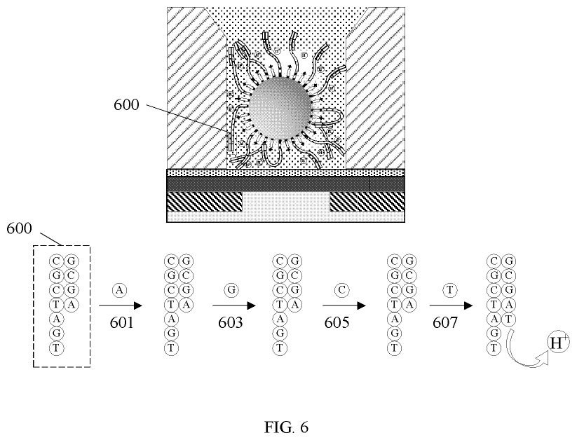

FIG. 6 illustrates a diagrammatic workflow of nucleic acid template sequencing performed by another exemplary nucleic acid sequencing apparatus according to various embodiments of the present disclosure. In particular, the fluidics delivering unit 104 may sequentially deliver a single type of dNTP into the reaction chamber in a pre-determined order, for example, in the order of dATP, dGTP, dCTP and dTTP. FIG. 6 also illustrates an enlarged portion of the growing duplex 600 formed on the surface of the microbead during the sequencing step. The next base on the template strand with the sequence of CGCTAGT waiting for polymerization is A. With the sequential addition of non-complementary dATP, dGTP and dCTP in steps 601, 603 and 605, respectively, the next base A at 3′ end of the sequencing primer remain unpolymerized. With the addition of complementary dTTP in step 607, a dTTP may be incorporated into the growing sequencing primer, accompanied with the release of a hydrogen ion as byproduct.

In accordance with the aforementioned embodiments, when each of the sensor well is connected with an ISFET sensor, the ion-sensitive layer of the ISFET sensor may detect and measure the concentration change of the hydrogen ions corresponding to the incorporation of the dNTPs at 3′ end of the sequencing primer (e.g., dTTP). In one embodiment, when a homopolymer region (e.g., poly(dA)) is present in the template, the incorporation of multiple dTTP molecules may result in a multi-fold signal change corresponding to the number of the repeatable bases in the template. For example, a homopolymer region including AA sequence repeats may cause a two-fold signal change compared to the signal generated by a single T in the template. The detection of hydrogen ions by the use of ISFET sensors may only require natural dNTPs, rather than dNTPs with different fluorescently labeled reversible terminators and complex fluorescent imaging platform, thereby significantly reducing the cost of the sequencing apparatus and reagent cost per run. Furthermore, the detection of hydrogen ions by the use of ISFET sensors may improve the sequencing efficiency. In one embodiment, the sequencing process may approximately take 60-90 minutes.

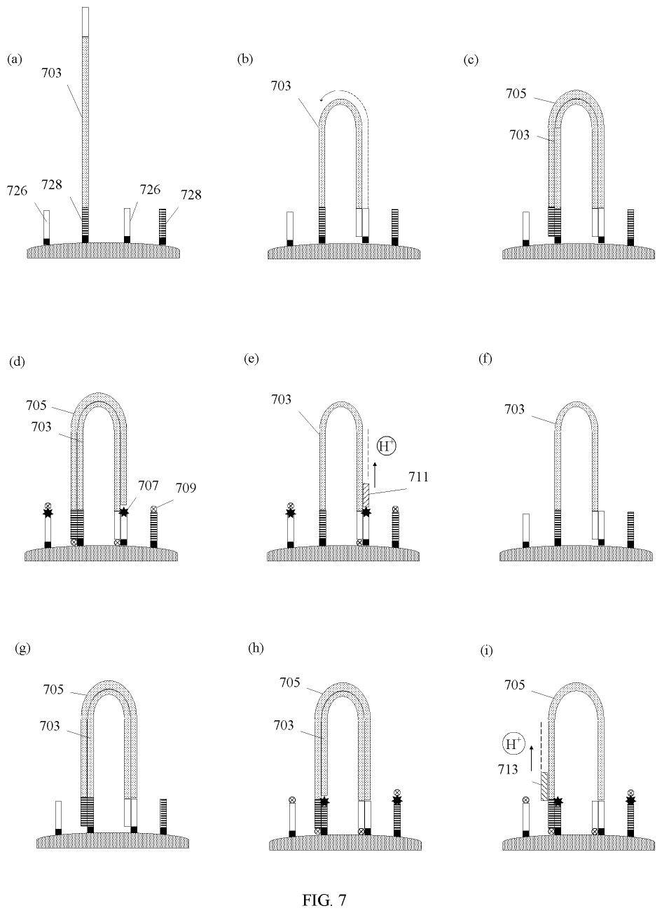

Alternatively, FIG. 7 illustrates a diagrammatic workflow of nucleic acid template amplification and sequencing performed by another exemplary nucleic acid sequencing apparatus according to various embodiments of the present disclosure. With reference to FIG. 7, the sequencing of the amplified nucleic acid template may be realized at both ends of the template sequence. For example, the nucleic acid template 703 immobilized on the surface of the microbead may bend over and annealed to the capturing oligo 726 (see (a) and (b) of FIG. 7). The capturing oligo 726 may extend to generate an elongated oligonucleotide strand 705. These steps may be repeated, thereby generating a plurality of double-stranded nucleic acid clones (see (c) of FIG. 7). Subsequently, the strand 705 may be cleaved by the cleavage reagent 707 and washed away after the denaturing process. The ends of the oligonucleotide 703 as well as the capturing oligos 726 and 728 may be blocked by the addition of ddNTPs 709 (see (d) and (e) of FIG. 7). With the addition of a sequencing primer 511, the sequencing may be started from 3′ end of the nucleic acid template clone 703. With the extension of the sequencing primer 511 in the presence of DNA polymerase and dNTPs, the incorporation of each dNTP into the elongated sequencing primer corresponding to the base of the nucleic acid template 703 may result in the release of one hydrogen ion as byproduct. Alternatively, the oligonucleotide strand 703 may be cleaved by the cleavage reagent and washed away after the denaturing process, while its complementary oligonucleotide strand 705 may be immobilized on the surface of the microbead. The ends of the oligonucleotide strand 705 as well as the capturing oligos 526 and 528 may be blocked by the addition of ddNTPs 709 (see (h) and (i) of FIG. 7). With the addition of another sequencing primer 713, the sequencing of the oligonucleotide strand 705 may be started from its 3′ end. As such, both ends of the nucleic acid template may be sequenced, enabling more accurate sequencing with higher efficiency compared with sequencing from a single-end of the template according to the aforementioned embodiments accompanied by FIG. 4.

As described in the aforementioned embodiments, when the sensor connected with each reaction well is an ISFET sensor, the ion-sensitive layer in the sensor may detect the concentration change of the hydrogen ion. In one embodiment, the release of the hydrogen ion may occur on the surface or in proximity to the ion-sensitive layer, ensuring the sensitivity and accuracy of the detection. In some embodiments of the present disclosure, the capturing, amplification and sequencing of the nucleic acid template may be performed on the surface of the microbeads. These microbeads may prevent the diffusion of the released hydrogen ions away from the bottom portion of the reaction well, or lateral diffusion into other reaction wells. Further, the microbeads may confine the elongated primer-template duplex in vicinity of the bottom of the reaction wells, such that with the further extension of the primer strand, the released hydrogen ions may be accurately detected by the ion-sensitive layer.

In another embodiment, the parameters of the reaction wells including volume and aspect ratio (e.g. the ratio between the diameter and depth of a reaction well) may be adjusted. For example, the depth of the reaction well may be increased such that the released hydrogen ions due to dNTP incorporation may be confined in the reaction well without lateral diffusion. In another embodiment, the determination of the parameters of the reaction wells may be in accordance with the microbeads disposed into the reaction wells. As such, each reaction well may include a single microbead, while the fluidic communication between the reaction chamber and the opening of the reaction well may not be influenced by the microbeads.

According to the aforementioned embodiments of the present disclosure, the exemplary apparatus and method for nucleic acid sequencing may provide a variety of advantages including high accuracy, efficiency, portability and affordability for users. For example, the digital capture of the nucleic acid templates on the surface of the microbeads may result in accurate and efficient amplification of the template to form a high density of nucleic acid template clones on the surface of the microbeads, which may further improve the accuracy of the following nucleic acid template sequencing. Furthermore, through the digital capture, the presence or absence of the template in each of the reaction wells may be easily determined through binary signal output. In addition, the time-consuming emulsion PCR may be avoided, such that the efficiency and accuracy of the genetic sequencing may further be improved.

In another embodiment, each of the reaction wells may be connected to an ISFET sensor which may detect the concentration change of the reactors or by-products corresponding to the nucleotide extension reactions. As such, natural reactors including dNTPs may be used in the nucleic acid sequencing apparatus and method, without the requirement of fluorescent-labeled reversible terminators or the use of complex fluorescent imaging platform. The size of the exemplary nucleic acid sequencing apparatus may be reduced, and because of the significant reduce in reagent cost, the sequencing expense per run may be more affordable to users. Furthermore, since the nucleic acid template is immobilized on the surface of the microbeads, it may be easier and more efficient for detecting the amplification of the template using ISFET sensors, thereby avoiding the use of emulsion PCR, or other probes/platforms (e.g., dyes and fluorescent imaging systems) for template amplification detection. In one embodiment, the nucleic acid sequencing apparatus and method may achieve a complete process from sample extraction/preparation to completion of sequencing in approximately 190-280 minutes.

Although the principles and implementations of the present disclosure are described by using specific embodiments in the specification, the foregoing descriptions of the embodiments are only intended to help understand the method and core idea of the method of the present disclosure. Meanwhile, a person of ordinary skill in the art may make modifications to the specific implementations and application range according to the idea of the present disclosure. In conclusion, the content of the specification should not be construed as a limitation to the present disclosure.

What is claimed is:

1. A method for nucleic acid sequencing, comprising:
providing a plurality of microbeads, wherein each of the plurality of microbeads is disposed in one of a plurality of reaction wells and modified with at least two capturing oligonucleotides with different sequences;
immobilizing a plurality of single-stranded nucleic acid templates on surfaces of the plurality of microbeads via annealing between the plurality of single-stranded nucleic acid templates and the at least two capturing oligonucleotides, wherein each single-stranded nucleic acid template includes two regions complementary to the different sequences of the at least two capturing oligonucleotides, respectively;
amplifying the immobilized plurality of single-stranded nucleic acid templates and producing a population of single-stranded nucleic acid template clones on the surfaces of the plurality of microbeads, wherein the population of single-stranded nucleic acid template clones is annealed with a plurality of sequencing primers;
sequentially disposing different types of nucleotide trisphosphates into the plurality of reaction wells wherein the different types of nucleotide trisphosphates are known, and detecting, by one or more ion-sensitive field-effect transistors (ISFETs), an ion concentration change in the plurality of reaction wells in response to incorporation of one of the different types of nucleotide trisphosphates at 3′ end of one of the sequencing primers, when the one of the different types of nucleotide trisphosphates is complementary to a corresponding nucleotide in the population of single-stranded nucleic acid template clones; and
sequencing the population of single-stranded nucleic acid template clones by repeatedly performing the sequentially disposing of the different types of nucleotide trisphosphates and the detecting, by the one or more ISFETs, of the ion concentration change in the plurality of reaction wells.

2. The method for nucleic acid sequencing according to claim 1, wherein:
a number of the plurality of single-stranded nucleic acid templates immobilized on a surface of each microbead via the annealing is less than or equal to a pre-determined value, wherein the pre-determined value is one.

3. The method for nucleic acid sequencing according to claim 1, wherein amplifying the immobilized plurality of single-stranded nucleic acid templates and producing the population of single-stranded nucleic acid template clones on the surfaces of the plurality of microbeads further comprise:
amplifying the immobilized plurality of single-stranded nucleic acid templates, thereby generating a plurality of double-stranded nucleic acid template clones on the surfaces of the plurality of microbeads;
denaturing the plurality of double-stranded nucleic acid template clones; and
producing the population of single-stranded nucleic acid template clones on the surfaces of the plurality of microbeads.

4. The method for nucleic acid sequencing according to claim 1, further comprising:
disposing a solution containing the plurality of single-stranded nucleic acid templates in the plurality of reaction wells, wherein a total number of the plurality of single-stranded nucleic acid templates in the solution is less than or equal to a total number of the plurality of reaction wells.

5. The method for nucleic acid sequencing according to claim 4, wherein:
the total number of the plurality of nucleic acid templates disposed into the plurality of reaction wells is less than or equal to 70% of the total number of the plurality of reaction wells.

6. The method for nucleic acid sequencing according to claim 1, further comprising:
determining a loading rate of the plurality of reaction wells, wherein the loading rate includes a ratio between a number of the reaction wells each containing one of the plurality of microbeads immobilized with one of the plurality of single-stranded nucleic acid templates and a total number of the plurality of reaction wells.

7. The method for nucleic acid sequencing according to claim 1, further comprising:
repeating the immobilizing of the plurality of single-stranded nucleic acid templates on the surfaces of the plurality of microbeads and the amplifying of the immobilized plurality of single-stranded nucleic acid templates.

8. The method for nucleic acid sequencing according to claim 7, wherein:
a loading rate is determined after each loading cycle including the immobilizing of the plurality of single-stranded nucleic acid templates on the surfaces of the plurality of microbeads and the amplifying of the immobilized plurality of single-stranded nucleic acid templates.

9. The method for nucleic acid sequencing according to claim 1, further comprising:
disposing a solution containing the plurality of microbeads into the plurality of reaction wells, wherein a total number of the plurality of microbeads in the solution is less than or equal to a total number of the plurality of reaction wells.

10. The method for nucleic acid sequencing according to claim 9, further comprising:
repeating the disposing of the solution containing the plurality of microbeads into the plurality of reaction wells.

11. A method for producing single-stranded nucleic acid template clones on a reaction well array, comprising:
providing the reaction well array including a plurality of reaction wells, wherein a plurality of microbeads is disposed in the plurality of reaction wells, and at least two capturing oligonucleotides with different sequences are immobilized on a surface of each of the plurality of microbeads;
adding a solution including a plurality of single-stranded nucleic acid templates into the plurality of reaction wells, wherein:
each of the single-stranded nucleic acid templates includes two regions complementary to the different sequences of the at least two capturing oligonucleotides, respectively,
the plurality of single-stranded nucleic acid templates is immobilized on surfaces of the plurality of microbeads via annealing between the nucleic acid templates and the at least two capturing oligonucleotides, and
a number of the single-stranded nucleic acid templates immobilized on a surface of each microbead via the annealing is less than or equal to a pre-determined value, and the pre-determined value is one; and
amplifying the immobilized plurality of single-stranded nucleic acid templates, thereby generating a plurality of double-stranded nucleic acid template clones; and
denaturing the plurality of double-stranded nucleic acid template clones and producing a population of single-stranded nucleic acid template clones on the surfaces of the plurality of microbeads.

12. The method according to claim 11, further comprising:
adding a solution containing the plurality of microbeads in the plurality of reaction wells.

13. The method according to claim 11, wherein:
a total number of the plurality of nucleic acid templates in the solution is less than or equal to a total number of the plurality of reaction wells.

14. The method according to claim 13, wherein:
the total number of the plurality of nucleic acid templates in the solution is less than or equal to 70% of the total number of the plurality of reaction wells.

15. The method according to claim 11, further comprising:
determining a loading rate of the plurality of reaction wells, wherein the loading rate includes a ratio between a number of the reaction wells each containing one of the plurality of microbeads immobilized with one of the plurality of single-stranded nucleic acid templates and a total number of the plurality of reaction wells.

16. The method according to claim 11, further comprising:
repeating the step of adding the solution including the plurality of single-stranded nucleic acid templates into the plurality of reaction wells and the step of amplifying the immobilized plurality of single-stranded nucleic acid templates.

17. The method according to claim 15, wherein:
the loading rate is determined by measuring, by one or more ion-sensitive field-effect transistors (ISFETs) configured to provide at least one output signal in response to a concentration or presence of one or more ions proximate thereto, ion concentration change corresponding to the amplification of the immobilized plurality of nucleic acid templates in the reaction wells, wherein the plurality of reaction wells is associated with the one or more ISFETs.

18. An apparatus for nucleic acid sequencing, the apparatus comprising:
a sensor array, including a plurality of ion-sensitive field-effect transistors (ISFETs) configured to provide at least one output signal corresponding to a concentration or presence of one or more ions proximate thereto;
a flow cell including an input, an output and a flow chamber, wherein the flow chamber is in fluidic connection with an opening of each reaction well of an array of reaction wells,
a fluidics delivering unit, configured to be in fluidic connection with the input of the flow cell, and configured to deliver at least one of the to-be-sequenced nucleic acid template and different types of known nucleotide trisphosphates, in a direction from the input to the output, to the reaction chamber, wherein:
a plurality of microbead is disposed in the array of reaction wells,
at least two capturing oligonucleotides with different sequences are immobilized on a surface of each of the microbeads, and the different sequences of the at least two capturing oligonucleotides are complementary to two regions of a to-be-sequenced nucleic acid template,
each of the reaction wells is associated with one of the plurality of ISFETs in the sensor array, and the one of the plurality of ISFETs is configured to provide the at least one output signal in response to ion concentration change in each of the reaction wells, and
the ion concentration change corresponds to incorporation of one of the different types of nucleotide trisphosphates at 3′ end of a sequencing primer annealed to the to-be-sequenced nucleic acid template, when the one of the different types of nucleotide trisphosphates is complementary to a corresponding nucleotide in the to-be-sequenced nucleic acid template.

19. The apparatus for nucleic acid sequencing according to claim 18, wherein:
the fluidics delivering unit is further configured to deliver a solution containing the plurality of microbeads.

20. The apparatus for nucleic acid sequencing according to claim 18, wherein:
the sensor array and the array of reaction wells are integrated on a same semiconductor chip.
